(19) United States
(12) Patent Application Publication
(10) Pub. No.: US 2005/0074428 A1
(43) Pub. Date: Apr. 7, 2005

(54) Adjuvant agent for hepatitis C

(76) Inventor: Wang-Sheng Ko, Taichung City (TW)

Correspondence Address: BIRCH STEWART KOLASCH & BIRCH, PO BOX 747, FALLS CHURCH, VA, 22040-0747, US

(21) Appl. No.: 10/755,468

(22) Filed: Jan. 13, 2004

(30) Foreign Application Priority Data
Oct. 7, 2003 (TW) 092127864

(51) Int. Cl. A61K 38/21; A61K 35/84; A61K 35/78

(52) U.S. Cl. 424/85.4; 424/195.15; 424/757

(57) ABSTRACT

An adjuvant agent used in combination with interferon and ribavirin for hepatitis C, containing 50 to 90 wt % cordyceps sinensis, and containing 10 to 50 wt % astragalus memsrancens.

### BACKGROUND OF THE INVENTION

1. Field of the Invention

The present invention relates to an adjuvant therapy for hepatitis C, which consists of a mixture of cordyceps sinensis and memsrancens astragalus, to enhance the effect of conventional therapy of interferon and ribavirin.

2. Description of the Prior Art

Hepatitis is a disease characterized by inflammation of the liver, usually producing swelling and, in many cases, permanent damage to liver tissues. A number of agents can cause hepatitis, including infectious diseases, chemical poisons, drugs and alcohol. Hepatitis C virus, also known as Non-A or Non-B hepatitis, is a RNA virus discovered in 1989. Hepatitis C is a contagious viral disease that leads to serious, permanent liver damage, and in many cases, death. Viral transmission is via bodily fluids and routine screening and examination decreases the risk of blood transfusion-related hepatitis C.

There are four types of hepatitis C virus, type I, II, III, and IV, based on the gene sequences. Among the hepatitis C patients in Taiwan, approximately 73 percent were infected by type II, 13 percent by type III, and 3 percent by type IV. Interferon therapy is used for type III and IV hepatitis, C, and is generally considered to be ineffective for type II and I infections.

The most recommended treatment for hepatitis C virus is the combination therapy of interferon and ribavirin, which is known to enhance the effect of interferon therapy. The virus can be removed by 18% after the combination therapy, and by 30% with an extended interferon therapy for one more year; however, the cost and side effects are the main concern for most patients. The common side effects of the interferon therapy include severe cold and flu-like symptoms such as fever, headache, muscle soreness, and nausea. These symptoms will disappear after two to three weeks; however, other side effects may appear the end stage of the interferon therapy. These include, tiredness, muscle soreness, leucopenia, anemia, dysphoria, weight loss, and hair loss. In addition, ribavirin is also known to cause side effects such as anemia, cough, tickle, rash, and insomnia.

### SUMMARY OF THE INVENTION

Due to the side effects, the cost, and the inefficiency of the interferon therapy as indicated above, the present invention is to provide an adjuvant therapy for ribavirin and interferon. The effect of adjuvant agent added to the combination therapy of interferon and ribavirin is two-fold. While the interferon kills hepatitis C virus, the complementary adjuvant agent serves to boost the immune system. This combination therapy with adjuvant agent was more effective than other therapies currently used for hepatitis C, particularly with cirrhosis, in which the level of IgG, IgA was decreased and serum complement concentration and natural killer cell, CD8⁺cell, CD4⁺cell, and CD4⁺/CD8⁺cell ratio were increased to illustrate the immune enhancing effect.

### DETAILED DESCRIPTION OF THE PREFERRED EMBODIMENTS COMPONENTS OF THE ADJUVANT AGENT

The major components of adjuvant agent are cordyceps sinensis and astragalus memsrancens, which are used in combination with the conventional therapy of interferon and ribavirin. This method improves the therapeutic efficacy of the conventional treatment for the hepatitis C. The constituents and pharmacology of the adjuvant agent are discussed as below:

Cordyceps sinensis consists of a variety of bioactive ingredients including amino acids, polysaccharides, cordycepin, cordycepic acid, ergosterol, nucleotide, superoxide dismutase, and trace elements. Among these constituents, nucleotide is considered the most important one because it is known to enhance the cell activity and the ability to against illness.

The pharmacology effects of cordyceps sinensis include antimicrobial, anti-inflammatory, antipyretics, antiarrhythmic, anti-aging, anti-tumor, immuno-stimulatory, and enhancing vascular smooth muscle tone. The cordyceps sinensis is known for its benefits for expectorants, cough, elderly illness, and promoting overall vitality and longevity. It is also used to treat diseases such as phthisis, anemia, angina pectoris, nephritis, hepatitis, cirrhosis, hypertension, diabetes mellitus, and several kinds of cancer. Previous studies have shown that cordyceps sinensis participates in cell-mediated or humoral immune response. The water extract of cordyceps sinensis is known to increase thymocytes mitosis and T lymphocytes proliferation. Moreover, the combination of cordyceps sinensis with persicae semen is used to treat severe posthepatitis cirrhosis by decreasing IgG, and IgA, increasing cell-lymphocyte rosette rate, and enhancing the efficiency of natural killer cell and CD4⁺/CD8⁺cell ratio.

Astragalus memsrancens, also know as milk-vetch root, is a perennial leguminosae herbaceous plant and is mainly cultivated in southern China. The root of astragalus memsrancens is used to prepare the extract for therapeutic purposes. For centuries, astragalus memsrancens has been one of the most commonly prescribed herbs by Chinese herbal doctors and it has always been considered a safe therapeutic agent with no toxic effects. By far, numerous bioactive constituents of astragalus memsrancens have been discovered, including polysaccharide, monosaccharide, flavonoid, alkaloid (such as choline and betaine), amino acids, glocuronic acid, folic acid, metallic elements (such as selenium), and diatomite materials. Astragalus memsrancens is known for its immunomodulatory effect and it is used to treat cancer and HIV patients by enhancing the immune system and regulating the interferon level in those patients. Recently, astragalus memsrancens is commonly used as a nutritional supplement as an alternative option for immuno-compromised patients who prefer traditional herbal medicine. Astragalus memsrancens increased the antibody production in spleen immune rosettes, including the progenitor cells of T cells and B cells. Furthermore, in clinical studies, astragalus memsrancens increased activity of cAMP in the spleen and IgG, IgA, IgM in the blood for both healthy persons and patients. Whereas in animal studies, it stopped the virus spreading by inducing γ-interferon.

Cordyceps sinensis is a general term used to describe the purified mycelium form of a variety of cultures isolated from hirsutella sinensis, paecilomyces sinensi, chrysosporium sinense, sporothrix insectorum, stachybotrys sp, tolypocladium, paecilomycse hepiali, or hirsutella hepialid. The cordyceps sinensis used in the present adjuvant agent is hirsutella sinensis. The hirsutella sinensis is the asexual form of cordyceps sinensis, which was identified as described in our previously submitted U.S. Pat. Nos. 6,251,606 and 6,271,003. Based on the ITS sequencing, hirsutella sinensis was chosen to represent cordyceps sinensis for their genetic similarity. Even through hirsutella sinensis was used in the present adjuvant agent, the other types of cordyceps sinensis, such as paecilomyces sinensi, chrysosporium sinense, sporothrix insectorum, stachybotrys sp, tolypocladium, paecilomycse hepiali, or hirsutella hepialid, are not excluded from the substitution of hirsutella sinensis as the component of the adjuvant agent for the same expected therapeutic efficacy to treat hepatitis C.

Moreover, cordyceps sinensis is commonly supplemented in health food, and although no side effects were observed in patients with hepatitis C, further toxicity investigation is mandatory and was conducted by National Research Institute of Chinese Medicine.

Animal Studies

Rats were administered by oral gavage with water extracts of Cordyceps sinensis mycelium or vehicle control once daily for 14 days. The amount of extract used in this study was 10 times higher than the recommended dose for human. The dependent variables of this study were listed below.

(1)Behavior (active contraction).

(2)Body weight and intake of food and water.

(3)Blood testing such as hemochrome, thrombocyte, red blood cell, hemoleukocyte, and clotting time.

(4) Blood chemistry such as bilirubin, AST, ALT, BUN, creatinine, glucose, total protein, albumin, K, Na, Ca.

(5)Urine chemistry such as settling rate, protein, electrolytic containing pH value.

(6) Organ biopsy such as heat, liver, lung, kidney, stomach, and cholecyst.

Among the dependent variables, including the body weight and intake of food and water, there is no significant difference between the treatment and the control groups. The levels of plasma BUN, plasma protein, and urine protein were increased by the treatment. However, the elevated urinary protein level does not suggest toxicity. Unlike in human, the urinary protein level in rats increases as the volume of plasma. Under the condition used in this experiment, the water extracts of Cordyceps sinensis mycelium is not toxic to rats.

Clinical Trials

Three double-blinded testing clinical trials was conducted to examine the effect of the proposed adjuvant agent in hepatitis C patients. Patients were treated with α-interferon with or without ribavirin for one week, and on the second week, the patients were randomly assigned into two groups. The two groups were treated with the adjuvant agent or placebo concurrently with α-interferon with or without ribavirin for 24 weeks and another 24 weeks along without α-interferon or ribavirin until the therapy was completed. Blood samples were collected and any side effects were documented throughout the experiment period. The dependent variables examined in this experiment were listed as follows.

1. Blood chemistry: WBC, Hemoglobin, Platelet, BUN, Creatinine, SGOT/SGPT, and HCV-RNA.

2. Occurrence of side effects

Experimental conditions and results of the clinical trials are described as follows:

The first clinical trial

A total 28 patients of hepatitis C were treated with Roferon-A (IFN) (3 MU) subcutaneously 3 times per week for 6 months. All patients recruited in this study have never been treated with Roferon-A. On the second week, the patients were randomly assigned into two groups treated with the adjuvant agent (N=10) or placebo (N=18) concurrently with Roferon-A till the treatment was completed.

Table 1 summarized the sustained virologic response (SVR) rate of the first clinical trail.

TABLE 1
SVR %

| | Time | | | |
|---|---|---|---|---|
| | | | 6 months | |
| | | | post | 12 months post |
| Group | 3 months | 6 months | the treatment | the treatment |
| IFN + | 4/10(40%) | 7/10(70%) | 7/10(70%) | 7/10(70%) |
| Adjuvant | | | | |
| agent | | | | |
| IFN + | 10/18(55.5%) | 6/18(33.3%) | 2/18(11.1%) | 2/18(11.1%) |
| Placebo | | | | |

Conclusion:

(1) The adjuvant agent enhanced the therapeutic effect of INF.

(2)The recurrence of hepatitis C was lower in the group treated with the adjuvant agent than the placebo group.

The second clinical trial

A total 20 patients of hepatitis C were treated with Roferon-A (IFN) (3 MU, three times a week) subcutaneously and ribavirin (1000 mg, twice a week) for 6 months. All patients recruited in this study have never been treated with Roferon-A or ribavirin. On the second week, the patients were randomly assigned into two groups treated with the adjuvant agent (N=8) or placebo (N =12) concurrently with Roferon-A and ribavirin till the treatment was completed.

Table 2 summarized the sustained virologic response (SVR) rate of the second clinical trail.

TABLE 2
SVR (%)

| | Time | | | |
|---|---|---|---|---|
| | | | 6 months | 12 months |
| | | | post | post |
| Group | 3 months | 6 months | the treatment | the treatment |
| Roferon-A and | 3/8(37.5%) | 7/8(87.5%) | 7/8(87.5%) | 7/8(87.5%) |
| ribavirin + | | | | |
| Adjuvant | | | | |
| agent | | | | |
| Roferon-A and | 0/12(0%) | 2/12(16.7%) | 2/12(16.7%) | 0/12(0%) |
| ribavirin + | | | | |
| Placebo | | | | |

Conclusion:

(1) The adjuvant agent enhanced the therapeutic effect of the combination therapy of INF and ribavirin.

(2) The recurrence of hepatitis C was lower in the group treated with the adjuvant agent than the placebo group. In the group treated with adjuvant agent, the SVR % remained zero (N=7) for two years after the treatment was completed

(3) Ribavirin did not appear to improve the therapeutic effect for hepatitis C in this study.

The third clinical trial

A total 32 patients of hepatitis C were treated with Roferon-A (IFN) (3 MU, three times a week) subcutaneously and ribavirin (1000 mg, twice a week) for 6 months. All patients recruited in this study have never been treated with Roferon-A or ribavirin. On the second week, the patients were randomly assigned into two groups treated with the ajuvant agent (N=16) or placebo (N=16) concurrently with Roferon-A and ribavirin till the treatment was completed.

Table 3 summarized the sustained virologic response (SVR) rate of the third clinical trail.

TABLE 3
SVR (%)

| | Time | | | |
|---|---|---|---|---|
| | | | 6 months | |
| | | | post | |
| Group | 3 months | 6 months | the treatment | Recurrence |
| Roferon-A and | 16/16(100%) | 16/16(100%) | 15/16(94%) | 1/16(6%) |
| ribavirin + | | | | |
| Adjuvant | | | | |
| agent | | | | |
| Roferon-A and | 10/16(63%) | 10/16(63%) | 3/16(19%) | 13/16(81%) |
| ribavirin + | | | | |
| Placebo | | | | |

TABLE 4
Adverse events observed in the adjuvant
agent-and placebo-treated group

| | Roferon-A and | Roferon-A |
|---|---|---|
| | ribavirin + Adjuvant | and ribavirin + |
| Adverse events | agent | Placebo |
| Fever, headache, myalgia | 83% | 90% |
| Fatigue | 75% | 5% |
| Arthralgia | 83% | 75% |
| Irregular bowl movement | 63% | 83% |
| Mild hair loss | 56% | 63% |
| Rash | 69% | 69% |
| Depression | 63% | 69% |
| Irritability | 13% | 19% |
| Insomnia | 5% | 83% |
| Weight loss | 56% | 90% |

Anemia

| | | |
|---|---|---|
| 10 g/dl < hemoglobin < 11 g/dl | 31% | 56% |
| 9 g/dl < hemoglobin < 10 g/dl | 6% | 13% |
| Hemoglobin < 9 g/dl | 6% | 0% |

Leukopenia

| | | |
|---|---|---|
| 3,000/UL < leukocytes < 3,500/UL | 19% | 31% |
| 2,500/UL < leukocytes < 3,000/U | 0% | 0% |
| Leukocytes < 2,500/UL | 19% | 6% |

Conclusion:

(1)The combination therapy of interferon, ribavirin, and the adjuvant agent increased the sustained virologic response rate and decreased the rate of relapse of hepatitis C.

(2)The adjuvant agent significantly decreased the occurrence of adverse events of interferon and ribavirin, such as irregular bowl movement, weight loss, anemia and leukopenia.

Based on the animal studies and clinical trials as described above, the proposed adjuvant agent improved the therapeutic effects and decreased the adverse events for hepatitis C patients treated with interferon and ribavirin.

In summary, the proposed adjuvant agent of the present invention comprises cordyceps sinensis and astragalus memsrancens, enhanced the therapeutic effects of interferon and ribavirin in hepatitis C patients. Furthermore, several trace elements were demonstrated to have beneficial effects in these patients. For example, zinc in adjuvant agent replenished the zinc lost in these patients; it also increased T cells production and enhanced the immunity of these patients.

1. An adjuvant agent used in combination with interferon and ribavirin for hepatitis C, the adjuvant agent comprising:
a. 50 to 90 wt % cordyceps sinensis; and
b. 10 to 50 wt % astragalus memsrancens.

2. The adjuvant agent for hepatitis C as claim 1, wherein the cordyceps sinensis used to prepare the adjuvant agent is isolated from the culture of asexual form of Hirsutella sinensis mycelium.

3. The adjuvant agent for hepatitis C as claim 1, wherein the cordyceps sinensis is purified from the mycelium which is consisted of a variety of cultures of paecilomyces sinensi, chrysosporium sinense, sporothrix insectorum, stachybotrys sp., tolypocladium sp., paecilomycse hepiali, and hirsutella hepiali.

4. The adjuvant agent for hepatitis C as claim 1, the amount of cordyceps sinensis, astragalus memsrancens is 70 to 80, 20-30 wt % of the adjuvant agent, respectively.

5. The adjuvant agent for hepatitis C as claim 1, wherein further comprising from 5 to 10 wt % zinc.

6. The adjuvant agent for hepatitis C as claim 5, the amount of cordyceps sinensis, astragalus memsrancens, and zinc is 70 to 80, 10-20, and 5-10 wt % of the adjuvant agent, respectively.
